(19) United States
(12) Patent Application Publication
(10) Pub. No.: US 2022/0249804 A1
(43) Pub. Date: Aug. 11, 2022

(54) Deflectable Sheath With Inflatable Balloon

(71) Applicant: Oscor Inc., Palm Harbor, FL (US)

(72) Inventors: Thomas P. Osypka, Palm Harbor, FL (US); Jorg TeBarek, Lingen (DE); Andrew J. Enerson, Hudson, FL (US); Chet Michael, Tampa, FL (US)

(21) Appl. No.: 17/728,596

(22) Filed: Apr. 25, 2022

Related U.S. Application Data
(63) Continuation-in-part of application No. 16/278,625, filed on Feb. 18, 2019, now abandoned.
(60) Provisional application No. 62/710,436, filed on Feb. 16, 2018.

(51) Int. Cl. A61M 25/00; A61M 25/01; A61M 25/10

(52) U.S. Cl. CPC A61M 25/0032; A61M 25/0147; A61M 2210/125; A61M 2205/103; A61M 25/10181

(57) ABSTRACT

A steerable intravascular catheter includes a handle assembly having opposed proximal and distal end portions and defining a longitudinal axis therebetween. An elongated sheath extending from the distal end portion of the handle assembly has opposed proximal and distal end portions, and includes a tubular body wall forming a central lumen for accommodating the introduction of a device and a fluid lumen radially outward from and parallel to the central lumen. The distal end portion of the elongated sheath is deflectable relative to the proximal end portion of the elongated sheath. A rotatable actuation assembly is associated with the handle assembly for controlling deflection of the distal end portion of the elongated sheath. An inflatable occlusion balloon is positioned on an outer surface of the distal end portion of the elongated sheath. The fluid lumen of the elongated sheath is in fluid communication with an interior of the balloon.

### CROSS-REFERENCE TO RELATED APPLICATIONS

This application is a continuation-in-part of U.S. patent application Ser. No. 16/278,625, filed on Feb. 18, 2019, now abandoned, which claims the benefit of and priority to U.S. Provisional Patent Application No. 62/710,436, filed Feb. 16, 2018, which are incorporated herein by reference.

### BACKGROUND OF THE INVENTION

### 1. Field of the Invention

The subject invention relates to intravascular catheters, and more particularly, to a guided intravascular catheter device having an inflatable balloon mounted on its distal end and a steering assembly for accurately placing the distal end of the sheath and balloon at a targeted location in a patient's body.

### 2. Description of Related Art

There are instances where physicians must introduce diagnostic and therapeutic devices into the body, such as diagnostic and therapeutic electrodes, ultrasound transducers and other surgical tools. The diagnostic and therapeutic devices are often carried by catheters, which allow physicians to gain access to the body in a minimally invasive manner by way of bodily lumens. In cardiac treatment, for example, a catheter is advanced through a main vein or artery into the region of the heart that is to be treated.

One method of introducing diagnostic and therapeutic devices into the body is to introduce a tubular member (typically a “catheter sheath”) into the vicinity of the targeted region. A diagnostic or therapeutic catheter device is then passed through the sheath to the targeted region. If necessary, the diagnostic or therapeutic catheter device may be removed after its function is performed, but the sheath can be left in place, so that other catheters or other devices can be advanced to the targeted region to complete the diagnostic and/or therapeutic procedure. One such device commonly advanced to the targeted region through the catheter sheath is a balloon occlusion catheter. Balloon occlusion catheters can be used to occlude vessels to temporary block up a vessel to then deploy contract media and or a drug to a certain location inside the human body or vascular system. Traditional balloon occlusion catheters can be introduced into the vascular system through a central lumen of the catheter sheath.

Catheter sheaths can be steerable. Examples of steerable sheaths and devices are disclosed in commonly assigned U.S. Pat. Nos. 9,498,602, 9,572,957 and 9,907,570 to Osypka et al. While these devices are well suited for the precise placement of diagnostic or therapeutic devices within a patient's body, these steerable sheath devices do not include a balloon for treatment.

There is a need, therefore, for an improved guiding sheath with a distally mounted inflatable balloon, which allows the distal section of the sheath to be deflected, is easy to navigate as a delectable guiding sheath, is efficient to fabricate and easy to use.

### SUMMARY OF THE INVENTION

A steerable intravascular catheter includes a handle assembly having opposed proximal and distal end portions and defining a longitudinal axis therebetween. An elongated sheath extends from the distal end portion of the handle assembly and has opposed proximal and distal end portions. The elongated sheath includes a tubular body wall forming a central lumen for accommodating the introduction of a device and a fluid lumen radially outward from and parallel to the central lumen. The distal end portion of the elongated sheath is deflectable relative to the proximal end portion of the elongated sheath. A rotatable actuation assembly is operatively associated with the handle assembly for controlling deflection of the distal end portion of the elongated sheath. An inflatable occlusion balloon is positioned on an outer surface of the distal end portion of the elongated sheath. The fluid lumen of the elongated sheath is in fluid communication with an interior of the balloon.

In accordance with some embodiments, the steerable intravascular catheter includes an inflation port positioned on the handle assembly in fluid communication with the fluid lumen for allowing the inflatable occlusion balloon to be inflated and deflated.

The elongated sheath can include a pull-wire lumen radially outward from and parallel to the central lumen. The steerable intravascular catheter can include an elongated pull-wire extending through the pull-wire lumen of the elongated sheath and terminating within the distal end portion of the elongated sheath. It is contemplated that the elongated pull-wire can have a proximal end operatively connected to the handle assembly and a distal end anchored to the distal end portion of the elongated sheath. In some embodiments, the steerable intravascular catheter includes a pull-wire anchor ring mechanically coupling a distal end of the elongated pull-wire to the distal end portion of the elongated sheath.

The distal end portion of the elongated sheath can be made from a softer material than the proximal end portion of the elongated sheath to accommodate deflection. The elongated sheath can define a circumference and a predetermined usable length (UL) extending from the proximal end portion of the elongated sheath substantially to the distal end portion of the elongated sheath. The predetermined UL can range from 30 cm to 120 cm.

The rotatable actuation assembly can include a rotatable control knob operatively connected to a proximal end of the elongated pull-wire. Rotation of the rotatable control knob can pull or release the elongated pull-wire and can cause the distal end portion of the elongated sheath to deflect away from the longitudinal axis or back toward the longitudinal axis. The handle assembly can include a drive mechanism for actuating the elongated pull-wire in response to bi-directional angular rotation of the rotatable control knob. Bi-directional angular rotation of the rotatable control knob about the longitudinal axis of the handle assembly can effectuate reciprocal axial movement of the elongated pull-wire and corresponding angular deflection of the distal end portion of the elongated sheath.

In accordance with some embodiments, the handle assembly can include a hemostatic valve operatively connected to the central lumen. The hemostatic valve is designed to minimize blood loss and prevent air embolisms. The handle assembly can include a luer type locking connection on a proximal end of the central lumen. The handle assembly can include a flush port in fluid communication with the central lumen to flush the central lumen. The proximal end portion of the elongated sheath can extend entirely through the handle assembly and terminate at a sealed access port communicating with the central lumen defined by the tubular body wall.

These and other features of the systems and methods of the subject disclosure will become more readily apparent to those skilled in the art from the following detailed description of the embodiments taken in conjunction with the drawings.

### BRIEF DESCRIPTION OF THE DRAWINGS

So that those skilled in the art to which the steerable intravascular catheter of the subject invention appertains will readily understand how to make and use the device without undue experimentation, preferred embodiments thereof will be described in detail herein below with reference to certain figures, wherein:

FIG. 1A is a schematic perspective view of a steerable intravascular catheter constructed in accordance with an embodiment of the subject invention, showing an inflatable occlusion balloon mounted on the distal end portion of an elongated sheath;

FIG. 1B is a schematic perspective view of the proximal end of the steerable intravascular catheter of FIG. 1A, showing the hemostatic valve;

FIG. 2 is a schematic cross-sectional view of the elongated sheath 1 for the steerable intravascular catheter illustrated in FIGS. 1 and 1A, showing the pull-wire lumen 2 and fluid lumen 3 on opposite sides of the central lumen 9;

FIG. 2A is an enlarged schematic cross-sectional view of the elongated sheath 1 shown in FIG. 2, but, for the sake of clarity, without the PTFE liner 15 for the central lumen 9 and without the pull-wire 4 in the pull-wire lumen 2;

FIG. 2B is an enlarged view of a portion of the elongated sheath 1 shown in FIG. 2A showing the partial elliptical shape of the inner surface 22A of the tubular body wall 22 adjacent to the pull-wire lumen 2; and

FIG. 2C is an enlarged view of a portion of the elongated sheath 1 shown in FIG. 2A showing the partial elliptical shape of the inner surface 22A of the tubular body wall 22 adjacent to the fluid lumen 3.

### DETAILED DESCRIPTION OF THE PREFERRED EMBODIMENTS

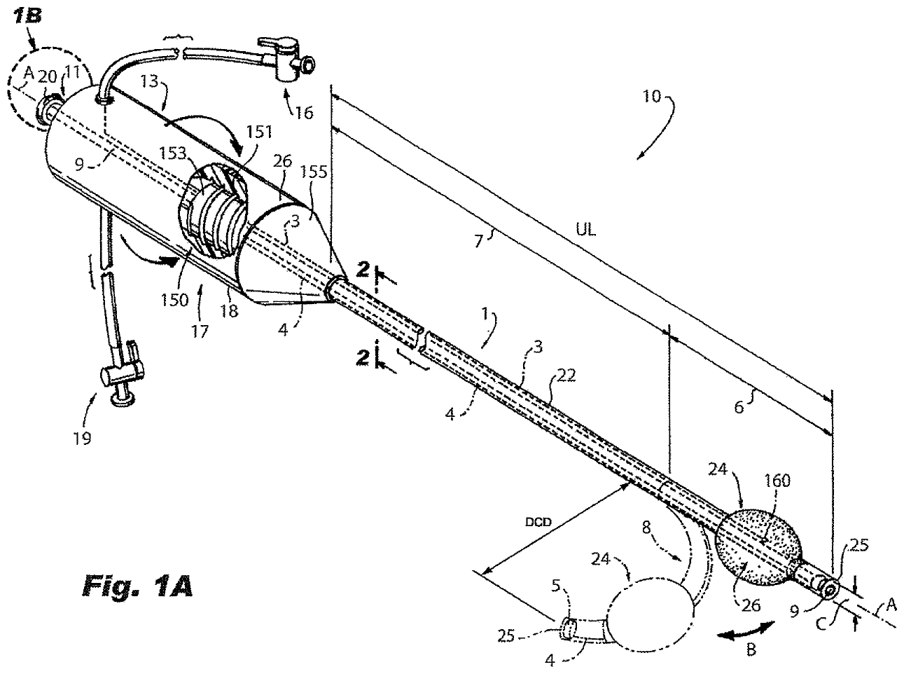

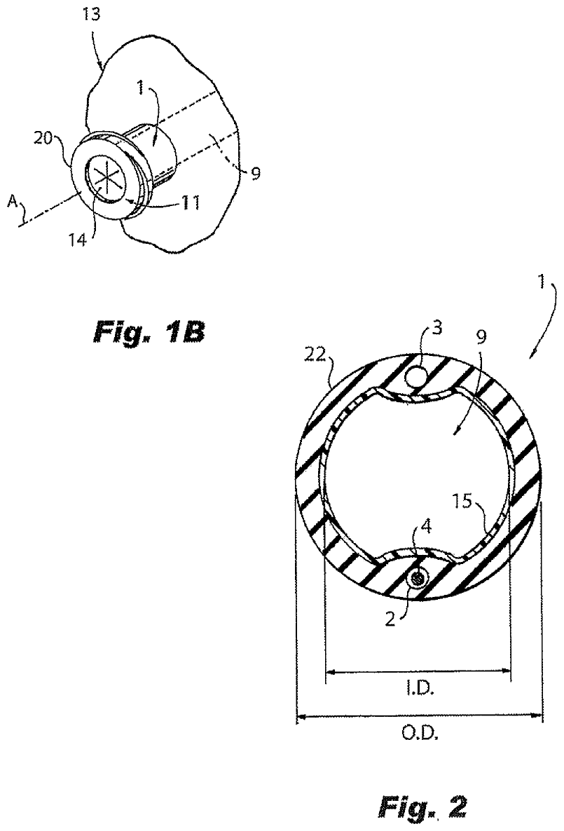

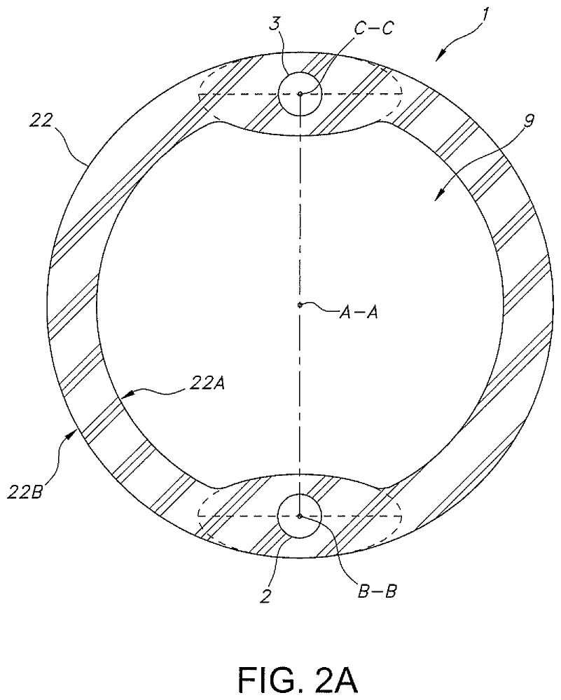

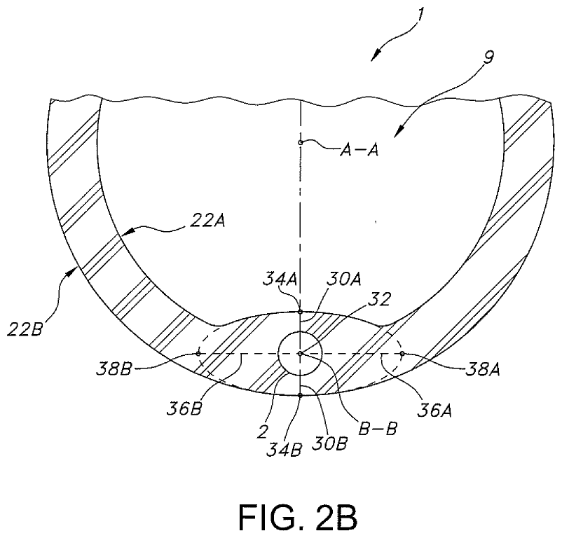

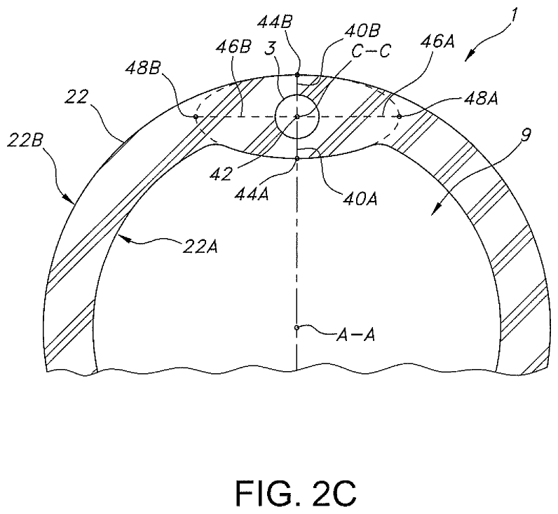

Reference will now be made to the appended drawings wherein like reference numerals identify similar structures or features of the subject invention. For purposes of explanation and illustration, and not limitation, there is illustrated in FIG. 1A a new and useful steerable intravascular catheter constructed in accordance with a preferred embodiment of the subject invention and designated generally by reference numeral 10. Other embodiments of the steerable intravascular catheter 10 in accordance with the disclosure, or aspects thereof, are provided in FIGS. 1B, 2 and 2A, as will be described. The steerable intravascular catheter 10 is adapted and configured to facilitate the intracardiac, renal and/or peripheral placement of diagnostic and therapeutic devices during a surgical procedure.

As shown in FIG. 1A, the steerable intravascular catheter 10 includes a handle assembly 13 having opposed proximal and distal ends defining a longitudinal axis A-A therebetween. An elongated sheath 1 extends from the distal end portion of handle assembly 13. The elongated sheath 1 has opposed proximal and distal end portions and includes a tubular body wall 22. The distal end portion 6 of the elongated sheath 1 is deflectable relative to the proximal end portion 7 of the elongated sheath 1. The deflectable distal end portion 6 of the elongated sheath 1 is made from a softer material than the proximal end portion (e.g. the stiffer sheath section 7) of the elongated sheath 1 to accommodate deflection. The handle assembly 13 includes a rotatable actuation assembly 17 for controlling deflection of the deflectable distal end portion 6 of the elongated sheath 1. An inflatable occlusion balloon 24 is positioned on an outer surface of the deflectable distal end portion 6 of the elongated sheath 1. The elongated sheath 1 defines a circumference C and a predetermined usable length (UL) extending from the start of the proximal end portion 7 of the elongated sheath 1 by the handle assembly 13 substantially to the distal most end of the distal end portion 6 of the elongated sheath 1. The predetermined UL can range from 30 cm to 120 cm.

Procedures such as the endovascular treatment of peripheral occlusions with mechanical aspiration/thrombectomy systems are made more efficient and easier to perform with the steerable sheath device 10. The combination of the elongated sheath 1, the mounted inflatable occlusion balloon 24, and the ability to mechanically deflect the distal tip portion 6 to appropriately steer the system into the correct target vessel allows for an increase in efficiency over traditional catheter sheaths.

As shown in FIGS. 1A, 2 and 2A, the tubular body wall 22 defines a central lumen 9 and a fluid lumen 3 radially outward from and parallel to the central lumen 9. The fluid lumen 3 of the elongated sheath 1 is in fluid communication with an interior 26 of the inflatable occlusion balloon 24. The fluid lumen 3 is schematically shown as a dashed line in FIG. 1A for the sake of clarity. Those skilled in the art will readily appreciate that the fluid lumen 3 is tubular shaped and extends within the tubular body wall 22 from a longitudinal position proximate to an inflation port 16, along the length of the elongated sheath 1, to a port 160 defined in the tubular body wall 22 within the interior 26 of the balloon 24. The inflation port 16 is positioned on the handle assembly 13 in fluid communication with the fluid lumen 3 allowing the inflatable occlusion balloon 24 to be inflated and deflated. Those skilled in the art will readily appreciate that a connecting tube or the like can extend from the fluid lumen 3 in the tubular body wall 22 to the inflation port 16. To inflate the balloon 24, an inflation fluid, such as saline solution or a contrast medium, is supplied to the interior 26 of the balloon 24 through the inflation port 16 using an inflation syringe, or the like. To deflate the balloon 24, the inflation syringe can provide a pulling vacuum to the interior 26 of the balloon 24 through the inflation port 16 and the balloon 24 returns to its deflated state.

With continued reference to FIGS. 1A, 2 and 2A, the elongated sheath 1 includes a pull-wire lumen 2 radially outward from and parallel to the central lumen 9. The steerable sheath device 10 includes an elongated pull-wire 4 extending through the pull-wire lumen 2 of the elongated sheath 1 and terminating within a distal end portion 6 of the elongated sheath 1. For sake of clarity, FIG. 1A only shows the elongated pull-wire 4, without the pull-wire lumen 2. Those skilled in the art will readily appreciate that, in the embodiment shown in the figures, the pull-wire lumen 2 has a tubular shape and extends within the tubular body wall 22 from a longitudinal position proximate a distal end of a manually rotatable control knob 18, described in more detail below, and down along the length of the elongated sheath 1 to a pull-wire anchor ring 5. The elongated pull-wire 4 is positioned within the pull-wire lumen 2 and has a proximal end that extends out of the pull-wire lumen 2 and is operatively connected to the handle assembly 13 and a distal end is anchored to the distal end portion 6 of the elongated sheath 1 at the pull-wire anchor ring 5. The pull-wire anchor ring 5 mechanically couples a distal end of the elongated pull-wire 4 to the distal end portion 6 of the elongated sheath 1. In the embodiment of FIG. 1A, the pull-wire anchor ring 5 is mounted proximate to a distal tip 25 of the distal end portion 6.

With continued reference to FIG. 1A, the manually rotatable control knob 18 of the rotatable actuation assembly 17 is operatively connected by way of a drive mechanism 150 to a proximal end of the elongated pull-wire 4. Manual rotation of rotatable control knob 18 pulls or releases the elongated pull-wire 4 by way of the drive mechanism 150, described below, and causes the distal end portion 6 of the elongated sheath 1 to deflect away from the longitudinal axis A-A or back toward the longitudinal axis A-A. The handle assembly 13 includes a drive mechanism 150 for actuating the elongated pull-wire 4 in response to bi-directional angular rotation of the rotatable control knob 18, as described in more detail below.

As shown in FIG. 1A, the drive mechanism includes a worm gear 153 mounted for reciprocal longitudinal movement within the interior cavity of the handle assembly 13 relative to the elongated sheath 1. The drive mechanism 150 further includes an axially rotatable drive nut 151 meshed with threads of the worm gear 153 for effectuating reciprocal longitudinal movement of the worm gear 153. The rotatable control knob 18 is directly connected to the drive nut 151 in the interior cavity of the handle assembly 13. The rotatable control knob 18 can be configured for gripping and rotation by a user to rotate the drive nut 150 and move the worm gear (e.g. work coil) 153. When the drive nut 150 is rotated by way of rotation of the rotatable control knob 18, the worm gear 153 rotates and moves longitudinally in either a distal or a proximal direction. A distal end portion 155 of the handle assembly 13 is fixed relative to the elongated sheath 1, such that the rotatable control knob 18 can be rotated with respect thereto.

With continued reference to FIG. 1A, in the handle assembly 13, the pull-wire 4 extends out of the tubular body wall 22 near a distal end 26 of the manually rotatable control knob 18 so that it can be coupled to the worm gear 153. The pull-wire 4 is coupled to the worm gear 153, e.g. coupled by way of a set screw, such that axial translation of the worm gear 153 pulls or releases the pull-wire 4 thereby causing deflection of the distal end portion 6. In FIG. 1A, the worm gear 153 is advanced to a distal position such that the worm gear 153 abuts the inner surface of the handle assembly 13 such that the worm gear 153 cannot be advanced further in the distal direction. This position can be associated with a straight condition of the sheath 1 (shown in solid lines). The worm gear 153 can be advanced proximally by rotation of the drive nut 151 to pull the pull-wire 4 and deflect the distal end portion 6 of the sheath 1 (as shown in the broken lines). The softer distal sheath end 6 in its deflected position is designated by numeral 8.

Bi-directional angular rotation of the rotatable control knob 18 about the longitudinal axis A-A of the handle assembly 13 effectuates reciprocal axial movement of the elongated pull-wire 4 and corresponding angular deflection of the distal end portion 6 of the elongated sheath 1, as shown schematically by the arcuate arrow B in FIG. 1A. Deflection of the distal end portion 6 can be defined by the deflection curve diameter (DCD), which can range from 7 mm to 50 mm. In some embodiments, the distal tip 25 of the distal end portion 6 can be deflected up to 180 degrees, or more. In other words, it can go from facing a distal direction to facing a proximal direction. While shown and described in conjunction with the drive mechanism 150, other suitable drive mechanisms can be used, e.g. those shown and described in commonly assigned U.S. Pat. Nos. 9,498,602, 9,572,957 and 9,907,570 to Osypka et al., which are hereby incorporated by reference in their entirety.

As shown in FIGS. 1A, 2 and 2A, the proximal end portion of the elongated sheath 1 extends entirely through the handle assembly 13 and terminates at a sealed access port 11 communicating with the central lumen 9 defined by the tubular body wall 22. The handle assembly 13 includes a hemostatic valve 14 operatively connected to the central lumen 9. The hemostatic valve 14 is designed to minimize blood loss and prevent air embolisms. The handle assembly 13 includes a luer type locking connection 20, e.g. fitting, on a proximal end of the central lumen 9. The handle assembly 13 includes a flush port 19 in fluid communication with the central lumen 9 to flush the central lumen 9. The central lumen 9 can include a PTFE liner 15. The tubular body 22 of the sheath 1 can have an outer diameter (OD) ranging from 6 to 30 French (F). An inner diameter (ID) of the tubular body 22 that defines, in-part, the central lumen 9 can range from 5 to 26 F. The hemostatic valve 14, luer type locking mechanism 20 and flush port 19 can be similar to those described in commonly assigned U.S. Pat. Nos. 9,498,602, 9,572,957, 8,974,420 and 9,907,570 to Osypka et al., all of which are hereby incorporated by reference in their entirety.

FIG. 2A is an enlarged schematic cross-sectional view of the elongated sheath 1 shown in FIG. 2, but, for the sake of clarity, without the PTFE liner 15 for the central lumen 9 and without the pull-wire 4 in the pull-wire lumen 2. As particularly shown, the tubular body wall 22 has an inner surface 22A spaced from an outer surface 22B by a thickness of the wall 22. The fluid lumen 2 resides in the thickness of the tubular body wall 22 and extends along a longitudinal axis B-B. Similarly, the pull-wire lumen 3 resides in the thickness of the wall 22 and extends along a longitudinal axis C-C.

FIG. 2B is an enlarged view of a portion of the elongated sheath 1 shown in FIG. 2A showing the partial elliptical shape of the inner surface 22A of the tubular body wall 22 adjacent to the pull-wire lumen 2. The partial elliptical shape comprises or is part of an ellipse that is depicted with dashed lines in the drawing and is defined by a semi-minor axis 30A that extends from a center point 32 coincident with the longitudinal axis B-B of the pull-wire lumen 2 to a vertex point 34A at the inner surface 22A and an opposed semi-minor axis 30B that extends from the center point 32 to a vertex point 34B at the outer surface 22B. An imaginary extension of the semi-minor axes 30A, 30B intersects the longitudinal axis A-A of the handle assembly 13 of the steerable intravascular catheter 10.

Opposed semi-major axes 36A and 36B of the ellipse comprising the partial elliptical shape of the inner surface 22A of the tubular body wall 22 adjacent to the pull-wire lumen 2 extend from the center point 32 at the longitudinal axis B-B to opposed vertex points 38A and 38B located between the inner and outer surfaces 22A and 22B of the tubular body wall 22. The opposed vertex points 38A and 38B are at a right angle or normal to the semi-minor axes 30A, 30B. As shown in the drawing, a major length of each of the semi-major axes 36A and 36B is at least 10% greater than a minor length of each of the semi-minor axes 30A, 30B.

FIG. 2C is an enlarged view of a portion of the elongated sheath 1 shown in FIG. 2A showing the partial elliptical shape of the inner surface 22A of the tubular body wall 22 adjacent to the fluid lumen 3. The partial elliptical shape comprises or is part of an ellipse that is depicted with dashed lines in the drawing and is defined by a semi-minor axis 40A that extends from a center point 42 coincident with the longitudinal axis C-C of the fluid lumen 3 to a vertex point 34A at the inner surface 22A and an opposed semi-minor axis 40B that extends from the center point 42 to a vertex point 44B at the outer surface 22B. An imaginary extension of the semi-minor axes 40A, 40B intersects the longitudinal axis A-A of the handle assembly 13 of the steerable intravascular catheter 10.

Opposed semi-major axes 46A and 46B of the ellipse comprising the partial elliptical shape of the inner surface 22A of the tubular body wall 22 adjacent to the fluid lumen 3 extend from the center point 42 at the longitudinal axis C-C to opposed vertex points 48A and 48B located between the inner and outer surfaces 22A and 22B of the tubular body wall 22. The opposed vertex points 48A and 48B are at a right angle or normal to the semi-minor axes 40A, 40B. As shown in the drawing, a major length of each of the semi-major axes 46A and 46B is at least 10% greater than a minor length of each of the semi-minor axes 40A, 40B.

The benefit of providing the tubular body wall 22 with a partial elliptical shape adjacent to at least one, and preferably both, of the pull-wire lumen 2 and the fluid lumen 3 is that there is an increased thickness to the wall 22 along the respective minor axes 30A, 30B and 40A, 40B in comparison to a conventional tubular body construction. This helps to improve the structural integrity of the wall adjacent to the pull-wire and fluid lumens 2, 3 so that the tubular wall has a robust construction that is less likely to rupture or fail during use.

While the steerable intravascular catheter device of the subject invention has been shown and described with reference to preferred embodiments, those skilled in the art will readily appreciate that various changes and/or modifications may be made thereto without departing from the spirit and scope of the subject disclosure.

What is claimed is:

1. A steerable intravascular catheter, comprising:
a) a handle assembly having opposed proximal and distal end portions and defining a longitudinal axis therebetween;
b) an elongated sheath extending from the distal end portion of the handle assembly and having opposed sheath proximal and distal end portions, wherein the sheath distal end portion is deflectable relative to the sheath proximal end portion, the elongated sheath including:
i) a tubular body wall having an inner wall surface spaced from an outer wall surface, wherein the inner wall surface defines a central lumen having a central lumen axis; and
ii) a first lumen in the tubular body wall, the first lumen having a first lumen axis that is radially outward from and parallel to the central lumen axis, wherein the inner wall surface adjacent to the first lumen has a first partial elliptical shape comprising a first ellipse with a first semi-minor axis of the first ellipse extending from a first center point coincident with the first lumen axis to the inner wall surface; and
c) a rotatable actuation assembly operatively associated with the handle assembly for controlling deflection of the sheath distal end portion.

2. The catheter of claim 1, wherein a first radial distance from the first lumen axis to the inner wall surface is equal to a second radial distance from the first lumen axis to the outer wall surface.

3. The catheter of claim 1, wherein the first ellipse comprises opposed first and second semi-major axes extending from the first center point to opposed first and second vertex points located in the tubular body wall between the inner and outer surfaces thereof.

4. The catheter of claim 1, further including a second lumen in the tubular body wall, the second lumen having a second lumen axis that is radially outward from and parallel to the central lumen axis, wherein the inner wall surface adjacent to the second lumen has a second partial elliptical shape comprising a second ellipse with a second semi-minor axis of the second ellipse extending from a second center point coincident with the second lumen axis to the inner wall surface.

5. The catheter of claim 4, wherein the second ellipse comprises opposed third and fourth semi-major axes extending from the second center point to opposed third and fourth vertex points located in the tubular body wall between the inner and outer surfaces thereof.

6. The catheter of claim 1, wherein the first lumen is a pull-wire lumen and the second lumen is a fluid lumen, or the first lumen is a fluid lumen and the second lumen is a pull-wire lumen.

7. A steerable intravascular catheter, comprising:
a) a handle assembly having opposed proximal and distal end portions and defining a longitudinal axis therebetween;
b) an elongated sheath extending from the distal end portion of the handle assembly and having opposed sheath proximal and distal end portions, wherein the sheath distal end portion is deflectable relative to the sheath proximal end portion, the elongated sheath including:
i) a tubular body wall having an inner wall surface spaced from an outer wall surface, wherein the inner wall surface defines a central lumen having a central lumen axis;
ii) a fluid lumen in the tubular body wall, the fluid lumen having a fluid lumen axis that is radially outward from and parallel to the central lumen axis, wherein the inner wall surface adjacent to the fluid lumen has a first partial elliptical shape comprising a first ellipse with a first semi-minor axis of the first ellipse extending from a first center point coincident with the fluid lumen axis to the inner wall surface and with opposed first and second semi-major axes of the first ellipse extending from the first center point to opposed first and second vertex points located in the tubular body wall between the inner and outer surfaces thereof; and
iii) a pull-wire lumen spaced from the fluid lumen in the tubular body wall, the pull-wire lumen being radially outward from and having a pull-wire axis that is parallel to the central lumen axis, wherein the inner wall surface adjacent to the pull-wire lumen has a second partial elliptical shape comprising a second ellipse with a second semi-minor axis of the second ellipse extending from a second center point coincident with the pull-wire axis to the inner wall surface and with opposed third and fourth semi-major axes of the second ellipse extending from the second center point to opposed third and fourth vertex points located in the tubular body wall between the inner and outer surfaces thereof, and
c) a rotatable actuation assembly operatively associated with the handle assembly for controlling deflection of the sheath distal end portion; and
d) an inflatable balloon positioned on an outer surface of the sheath distal end portion, wherein the fluid lumen of the elongated sheath is in fluid communication with an interior of the balloon.

8. The catheter of claim 7, further comprising an inflation port positioned on the handle assembly in fluid communication with the fluid lumen allowing the inflatable balloon to be inflated and deflated.

9. The catheter of claim 7, wherein the pull-wire lumen is diametrically opposed to the fluid lumen across from the central lumen.

10. The catheter of claim 7, further comprising an elongated pull-wire extending through the pull-wire lumen of the elongated sheath and terminating within the sheath distal end portion.

11. The catheter of claim 10, wherein the elongated pull-wire has a proximal end operatively connected to the handle assembly and a distal end anchored to the sheath distal end portion.

12. The catheter of claim 10, wherein the rotatable actuation assembly includes a rotatable control knob operatively connected to a proximal end of the elongated pull-wire, and wherein rotation of the rotatable control knob pulls or releases the elongated pull-wire to cause the sheath distal end portion to deflect away from the longitudinal axis or back toward the longitudinal axis.

13. The catheter of claim 12, wherein the handle assembly includes a drive mechanism for actuating the elongated pull-wire in response to bi-directional angular rotation of the rotatable control knob, and wherein bi-directional angular rotation of the rotatable control knob about the longitudinal axis of the handle assembly effectuates reciprocal axial movement of the elongated pull-wire and corresponding angular deflection of the sheath distal end portion.

14. The catheter of claim 7, wherein the sheath distal end portion is made from a softer material than the sheath proximal end portion to accommodate deflection.

15. The catheter of claim 7, wherein the elongated sheath defines a circumference and a predetermined usable length (UL) extending from the sheath proximal end portion substantially to the sheath distal end portion, and wherein the predetermined UL ranges from 30 cm to 120 cm.

16. The catheter of claim 7, wherein the handle assembly includes a hemostatic valve operatively connected to the central lumen.

17. The catheter of claim 7, wherein the handle assembly includes a luer type locking connection at a proximal end of the central lumen.

18. The catheter of claim 7, wherein the handle assembly includes a flush port in fluid communication with the central lumen.

19. The catheter of claim 7, wherein the sheath distal end portion extends entirely through the handle assembly and terminates at a sealed access port communicating with the central lumen defined by the tubular body wall.

20. The catheter of claim 7, wherein a diameter of the central lumen is larger than a diameter of the fluid lumen.
